(19) United States
(12) Patent Application Publication
(10) Pub. No.: US 2006/0135841 A1
(43) Pub. Date: Jun. 22, 2006

(54) High dose rate device

(75) Inventor: Eric van't Hooft, Brasschaat (BE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Isodose Control B.V., Veenendaal (NL)

(21) Appl. No.: 11/144,274

(22) Filed: Jun. 3, 2005

(30) Foreign Application Priority Data
Jun. 3, 2004 (NL) 1026323

(51) Int. Cl. A61N 5/00

(52) U.S. Cl. 600/3

(57) ABSTRACT

A device is described for storing and manipulating a radiation source, comprising a safe for storing at least one capsule comprising a radioactive material, a transport element for moving the capsule through a transport tube connected to the safe and leading to an applicator, and a drive mechanism for driving the transport element. According to the invention, the capsule comprises an enriched low energy radiation source for emitting gamma radiation in the range of 10-100 keV. For this purpose, a source is utilized having a sufficiently high specific activity for performing a high does rate (HDR) radiation treatment.

### CROSS-REFERENCE TO RELATED APPLICATION

This application claims priority of Netherlands Application NL 1026323, filed on Jun. 3, 2004, the contents of which are expressly incorporated herein by reference in their entirety including the contents and teachings of any references contained therein.

### BACKGROUND OF THE INVENTION

This invention relates to devices for storing and manipulating a radiation source, comprising a safe for storing at least one capsule comprising a radioactive material, a transport element for moving the capsule through a transport tube connected to the safe and leading to an applicator, and a drive mechanism for driving the transport element.

With such devices, through the drive mechanism, for instance a transport wire wound onto a reel and connected with the capsule, a source can be brought to an applicator and halted or moved at one or more sites to locally apply concentrated radiation in the body of a patient. Such devices are known and comprise a radioactive source of the isotope Cobalt 60, Cesium 187 or Iridium 192, which can be generally designated as “strong radiators.”

Such isotopes require a highly shielded, 30-75 cm concrete treatment chamber to protect hospital personnel and other attendants.

### BRIEF SUMMARY OF THE INVENTION

The invention comprises a device with an isotope having a considerably lower emitted radiation energy, thereby allowing work to be done in standard treatment rooms or standard operating rooms with a shielding of about 25 cm concrete at most.

This is achieved, according to the invention, by the use of the features of the claimed invention. A lower energy radiation source is used which has as an advantage that the shielding measures are considerably less stringent because of the lesser penetrative power of the lower energy radiator. By enriching the low energy radiation source, a higher dose rate can be delivered and can shorten a treatment without the radiation source unduly increasing in volume. It is noted that in itself the low energy radiation source as an application is known in the treatment of tumors, but the intensity of these radiators is too low to use a so called high dose rate treatment (HDR treatment), whereby a tumor is exposed for a relatively short time to a high radiation intensity. In view of the specific use of the device, the known low energy radiators would necessitate an unduly long radiation time to deliver a sufficient dose.

Preferably, therefore, an isotope is used having a relatively lower energy with respect to the above-mentioned sources, such as Ytterbium-169, Iodine-125, Palladium-103, or Thulium-170 or Tungsten-181. According to the invention, these sources are enriched to a sufficient concentration to obtain sufficiently specific activity in a small capsule. This can be done, for instance, through an ultracentrifuge process.

In an embodiment, the source has been enriched such that a delivered dose rate comes to lie in the HDR range. This range is defined in the so-called ICRU reports (ICRU-38 en ICRU-58). In a specific embodiment, the source is enriched such that a dose rate is at least 12 Gray/hour in a clinical specification spot, for instance at a distance of about 1 cm from the source.

In an embodiment, the source comprises Palladium-103. The weight percentage of Palladium-103 is then at least 3%. In another embodiment, the source comprises Iodine-125, in a weight percentage of at least 15%. In a further embodiment, the source comprises Ytterbium-169, in a weight percentage of Ytterbium-169 of at least 0.5%. In another embodiment, the source comprises Tungsten-181 that is enriched to a weight percentage of at least 5%.

The capsule in an exemplary embodiment has a diameter of at most 5 mm and a length of at most 50 mm.

### BRIEF DESCRIPTION OF THE SEVERAL VIEWS OF THE DRAWING(S)

The appended claims set forth the features of the present invention with particularity. The invention, together with its objects and advantages, may be best understood from the following detailed description taken in conjunction with the accompanying drawings of which:

FIG. 1 shows a schematic set-up of the device;

FIG. 2 shows a schematic cross section of the source; and

FIG. 3 shows the results of a simulation of a dose absorbed by tissue.

### DETAILED DESCRIPTION OF THE INVENTION

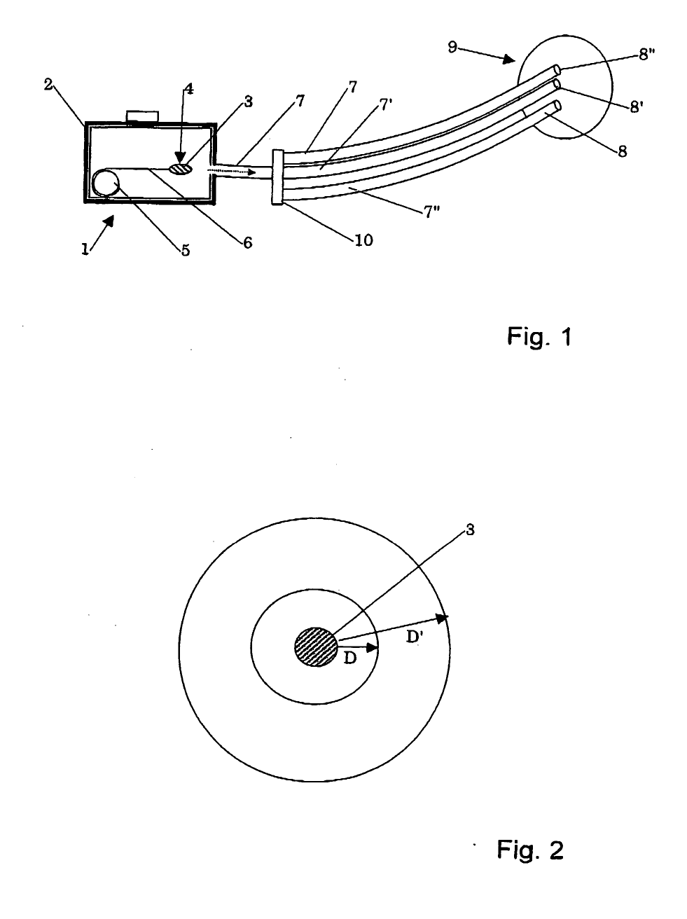

In FIG. 1 there is schematically represented an HDR device 1 provided with a safe 2 for storing at least one capsule 4 comprising a radioactive source 3, a transport wire 6 wound onto a reel 5 and connected with the capsule 4, to bring the capsule 4 via a transport tube 7 to an applicator 8 which has been introduced into tissue 9 to be irradiated. Such an HDR device is a device whereby tissue is irradiated by one or more radiation sources with a relatively high activity, so that in a short time a high does of radiation is absorbed by the tissue. According to the example, the capsule 4 has a diameter of at most 2.5 mm, preferably about 1 mm, for easy introduction via a tube into the body of a patient. For the types of radiations contemplated, greater diameters, while possible, are usually too great to allow positioning in the body without undesirable damage. Recommended with a view to easy transport of the capsule 4 is a length of at most ca. 50 mm, preferably even less, ca. 5 mm. As a consequence of this dimensioning of the capsule 4, the source 3 must have a sufficiently specific activity (i.e. a delivered radiation power per volume) to be effective for a radiation treatment. Too low a specific activity results in too long a treatment duration or can only be compensated by an amount of radiation material too large to be introduced into the tissue 9 via the transport tube 7. FIG. 1 depicts a version of the device 1 in which a switch 10 is provided for connecting one or more transport tubes 7, 7′, 7″ simultaneously, which can be connected to one or more applicators 8, 8′, 8″. This enables simultaneous displacement of several capsules which may or may not be filled with radioactive material.

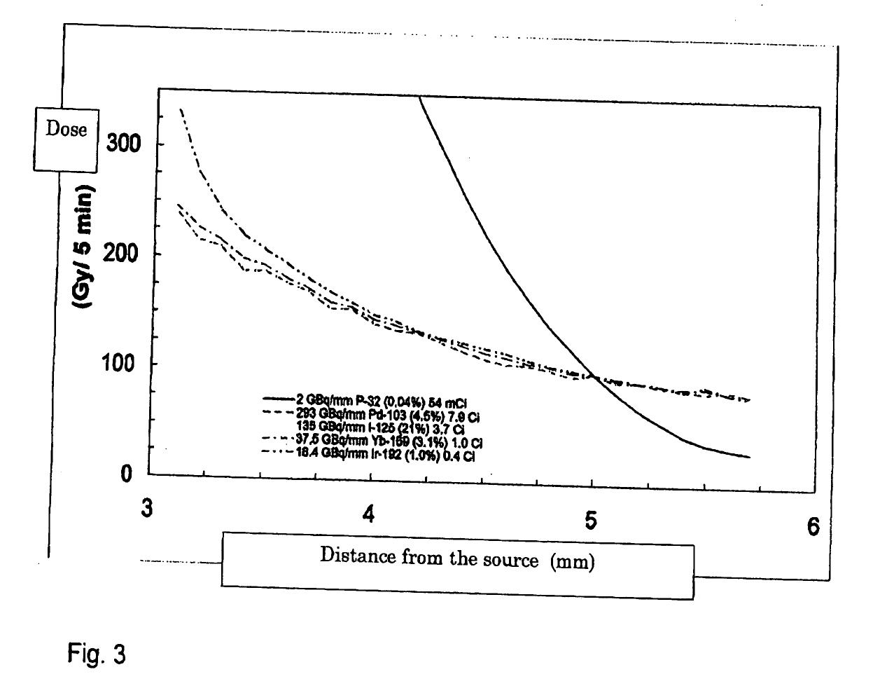

FIG. 2 schematically shows the simulation set-up for the table of FIG. 3, with the radiation source comprising a cylinder-shaped capsule having a diameter of 3 mm. At distances D, D′, each time a total absorbed dose per 5 minutes has been calculated over the total circumference of the cylinder (see FIG. 3). A practical measure that is used for a desired radiation level for irradiating tissue is 50 Gray, while values of 10 Gray or more can be effective. The table in FIG. 3 shows the absorption values found for radiation sources enriched with different low-energy radiators, understood to include in particular a gamma radiator in the energy range of 10-100 keV.

To achieve a desired absorption level, the table shows that for Palladium-103, with a dose rate of 29 GBq/cm, a weight percentage of 4.5% is desired. Likewise, it has been found that for Iodine-125 (135 GBq/mm) a minimum weight percentage of 21% is desired, and for Ytterbium-169 (37.5 GBq/mm) a weight percentage of 2.5%. Finally, for Iridium-192 a desired weight percentage of 1% has been found.

The invention is not limited to the isotopes mentioned in the specification. It is also possible to use other isotopes of a sufficiently low energy, so that they can easily be shielded and they achieve a sufficiently high specific activity to fit into a small capsule. Such sources are understood to fall within the scope of the claims as defined in the following.

What is claimed is:

1. A device for storing and manipulating a radiation source, comprising:
a safe for storing at least one capsule comprising a radioactive material;
a transport element for moving the at least one capsule through a transport tube connected to the safe and leading to an applicator; and
a drive mechanism for driving the transport element; characterized in that the at least one capsule comprises a high dose rate (HDR) radiation source for emitting radiation substantially in a range of 10-100 keV.

2. A device according to claim 1, wherein the HDR radiation source is enriched such that a delivered dose rate is within the HDR range.

3. A device according to claim 1, characterized in that the HDR radiation source comprises Palladium-103.

4. A device according to claim 3, characterized in that a weight percentage of Palladium-103 is at least 3%.

5. A device according to claim 1, characterized in that the HDR radiation source comprises Iodine-125.

6. A device according to claim 5, characterized in that a weight percentage of Iodine-125 is at least 15%.

7. A device according to claim 1, characterized in that the HDR radiation source comprises Ytterbium-169.

8. A device according to claim 7, characterized in that a weight percentage of Ytterbium-169 is at least 0.5%.

9. A device according to claim 1, characterized in that the HDR radiation source contains Thulium 170 (TM¹⁷⁰).

10. A device according to claim 1, characterized in that the HDR radiation source contains Tungsten 181 (W¹⁸¹).

11. A device according to claim 10, characterized in that the weight percentage of Tungsten 181 (W181) is at least 5%.

12. A device according to claim 1 characterized in that the HDR radiation source has a diameter of at most 5 mm.

13. A device according to claim 1 characterized in that the HDR radiation source is at most 20 mm long.

14. A device according to claim 1 characterized in that the device is provided with a switch for simultaneously connecting one or more transport tubes which are connected to one or more applicators.

15. A device according to claim 1, characterized in that the device is provided with a switch capable of simultaneously moving several capsules which may or may not be filled with radioactive material.

16. A device according to claim 1 characterized in that the device has several capsules filled with different radioactive materials.
